(19) United States
(12) Patent Application Publication
(10) Pub. No.: US 2019/0307426 A1
(43) Pub. Date: Oct. 10, 2019

(54) IMAGING SYSTEM USING RECEPTOR-TARGETED MICROBUBBLES

(71) Applicant: ARIZONA BOARD OF REGENTS ON BEHALF OF THE UNIVERSITY OF ARIZONA, Tucson, AZ (US)

(72) Inventors: Bhaskar Banerjee, Tucson, AZ (US); Terry O. Matsunaga, Tucson, AZ (US); Josef Vagner, Tucson, AZ (US); Khanh Kieu, Tucson, AZ (US)

(21) Appl. No.: 16/315,467

(22) PCT Filed: Jul. 7, 2017
(86) PCT No.: PCT/US17/41124

§ 371 Date: Jan. 4, 2019

Related U.S. Application Data
(60) Provisional application No. 62/359,454, filed on Jul. 7, 2016.

(51) Int. Cl. A61B 8/08; A61B 8/14; A61K 9/113; A61K 9/127; A61K 9/14; A61B 5/00; A61K 9/107; A61K 9/50; A61B 8/00

(52) U.S. Cl. CPC A61B 8/481; A61B 8/14; A61K 9/113; A61K 9/127; A61K 9/14; A61B 8/085; A61B 8/4416; A61K 9/1075; A61K 9/5015; A61B 5/0066; A61B 5/0068; A61B 5/0073; A61B 5/0071

(57) ABSTRACT

A system and method that uses receptor-targeted microbubbles to locate abnormal cell tissue for therapy is disclosed. The method includes applying receptor-targeted microbubbles to abnormal cell tissue; imaging the applied microbubbles using an imaging system; and locating the abnormal cell tissue using the imaged, applied microbubbles, wherein the imaging system detects and 5 transmits imaging information of the applied microbubbles through a gaseous environment, i.e., a non-contact procedure. Embodiments of the system and method combine the targeting of microbubbles to abnormal cell tissue and an imaging system, the combination of which is capable of accurately and rapidly assessing the surgical margin for presence of unseen cancer in, for example, the operating room, at bedside, or in an 10 office, within or outside the body.

### CROSS REFERENCE TO RELATED APPLICATION(S)

This application claims priority to U.S. provisional patent application No. 62/359,454, filed on Jul. 7, 2016, which is hereby incorporated herein by reference in its entirety.

### GOVERNMENT SPONSORSHIP

This invention was made with government support under Grant No. P50 CA095060 awarded by NIH. The government has certain rights in the invention.

### FIELD OF THE INVENTION

Embodiments are in the field of imaging systems and methods for using same for therapy. More particularly, embodiments disclosed herein relate to systems and methods of using receptor-targeted microbubbles to locate abnormal cell tissue for therapy.

### BACKGROUND OF THE INVENTION

One fourth of all deaths in the United States are caused by cancer. The diagnosis of cancer patients relies greatly on the time and accuracy of the detection of cancerous (or abnormal cell tissue) sites in the body. If the diagnosis can be made during the early stages of disease progression, then the prognosis for longer-term survival and even cure can be greatly increased. In addition to early diagnosis, there is also a need to accurately localize and stage disease for appropriate therapy as well as a further demand to assess effectiveness of that treatment (Theranostics). The aforementioned can potentially be achieved through the detection of both cancerous tissues and inflammatory markers in the body and deliver targeted therapy to the identified diseased tissue. Furthermore, with respect to surgical resections of cancerous tissue, a fast and reliable method for determining cancer-free margins intraoperatively that avoids the high sampling error associated with current methods of frozen-section microscopy of blind biopsies is of high priority.

In the past three decades, lipid microbubbles have been extensively explored as contrast agents to enhance ultrasound echoes in applications ranging from echocardiography to molecular imaging of vascularized tumors with a high degree of sensitivity. The diagnostic capability of lipid microbubbles (1-10 μm) for contrast-enhanced ultrasound (CEUS) is well established as an inexpensive and sensitive tool that provides both anatomical and functional information of tissue in real time. A new technique, not yet approved in the clinical setting, is the use of targeted microbubbles as a diagnostic tool. These ligand-conjugates decorated on the outer surface of microbubbles are designed for selectivity to an individual cell type over normal cells due to the overexpression of specific receptor proteins the surface of these cells. This development of targeted microbubbles could lead to additional diagnostic applications for CEUS as well as other imaging modalities such as; 1) early detection of cancerous lesions or other abnormal cell tissue; 2) localization of inflammation (including abnormal cell tissue); and 3) an especially large role in emerging theranostics.

The removal of all tumor mass at surgery is a challenge as not all the cancer can be seen with the naked eye. Clean surgical margins are difficult to determine macroscopically as flat areas are indistinguishable from normal tissue, and microscopy of a large surface or margin is impractical and time-consuming. Satellite lesions are also easily missed when removing the main tumor bulk. If cancer remains following the completion of surgery, recurrence is certain with a poorer prognosis. Embodiments herein provide a solution by using (e.g., lipid) microbubbles decorated with molecules (such as peptides or antibodies that bind to tumor or abnormal cell tissue receptors). Once the targeted microbubbles attach to the tumor/abnormal cells, the gas-filled bubbles can be easily imaged (using a variety of imaging techniques, such as ultrasound, optical coherence tomography, confocal microscopy, or endoscopy, or camera) and the presence of cancer can easily be visualized and distinguished from benign areas. This method is: a) rapid; b) specific; and c) provides a macroscopic image using a process that is accurate at a microscopic, cellular level.

However, during the surgical resection of certain cancers, a large amount of tissue surrounding the tumor cannot be excised but it is equally important to remove all the cancer at the time of surgery. Examples include cancer of the pancreas, bile duct, rectum, brain, and breast. This method will enable the surgeon to confirm a clean margin at the time of surgery, as well as help detect and remove or treat any satellite (cancerous or dysplastic) lesions, without resecting large amount of normal/healthy tissue. The method may be theranostic and may include bubbles as mediators of therapy.

The use of receptor-targeted lipid microbubbles imaged by ultrasound is a novel method of detecting and localizing disease. However, since ultrasound requires a medium between the transducer and the object being imaged, it is impractical to apply to an exposed surface in a surgical setting where sterile fields need be maintained and, moreover, ultrasound gel may cause the bubbles to collapse. Multiphoton microscopy (MPM) (or Multiphoton imaging (MPI)) is an emerging tool for accurate, label-free imaging of tissues and cells with high resolution and contrast. The inventors have recently determined a novel method for detecting targeted microbubble adherence to the upregulated plectin-receptor on pancreatic tumor cells using MPM/MPI. Specifically, the third-harmonic generation response can be used to detect bound microbubbles to various cell types presenting MPM/MPI as an alternative and useful imaging method. This is a novel technique that can potentially be translated as a diagnostic tool for the early detection of cancer and inflammatory disorders.

Ultrasound is the traditional method to image microbubbles. However, MPI does not require a liquid or gel interface between the detector and tissue surface, making it suitable for the operating room.

Current methods involve analyzing a frozen section biopsy for cancerous cells at the time of surgery, but this limits sampling to a tiny fraction of the entire surgical margin; microscopic examination of the entire resected specimen, including the surgical margin is only possible several days post-surgery, when it is too late to re-do the operation if cancer is found at the margin on the final pathological examination.

Thus, it is desirable to provide embodiments of a system and method of using receptor-targeted microbubbles to locate abnormal cell tissue for therapy that do not suffer from the above drawbacks.

These and other advantages of the present invention will become more fully apparent from the detailed description of the invention herein below.

### SUMMARY OF THE INVENTION

Embodiments are directed to a method of using receptor-targeted microbubbles to locate abnormal cell tissue for therapy. In an embodiment, the method comprises: applying receptor-targeted microbubbles to abnormal cell tissue; imaging the applied microbubbles using an imaging system; and locating the abnormal cell tissue using the imaged, applied microbubbles, wherein the imaging system detects and transmits imaging information of the applied microbubbles through a gaseous environment (or contactless procedure).

In an embodiment, the imaging is performed by the imaging system using a light source (such as a laser).

In an embodiment, the imaging is performed by the imaging system using a multi-photon imaging (MPI) technique.

In an embodiment, the imaging is performed by the imaging system using a technique selected from the group consisting of multi-photon imaging (MPI), optical coherence tomography (OCT), ultrasound, and a combination thereof.

In an embodiment, the abnormal cell tissue is located adjacent to healthy pancreatic tissue.

In an embodiment, the abnormal cell tissue is located adjacent to healthy pancreatic, healthy brain, or other type of healthy tissue.

In an embodiment, the abnormal cell tissue is located below the surface of healthy tissue.

In an embodiment, the abnormal cell tissue is either in-vivo or ex-vivo.

In an embodiment, the imaging of the applied microbubbles is performed without the use of labels or markers.

In an embodiment, the imaging of the applied microbubbles is performed using harmonics (e.g., third-order harmonics).

In an embodiment, the method further comprises applying therapy to the abnormal cell tissue. The imaging and applying of therapy may be performed substantially simultaneously.

Embodiments are also directed to a system that uses receptor-targeted microbubbles to locate abnormal cell tissue for therapy. In an embodiment, the system comprises: an application system that applies receptor-targeted microbubbles to abnormal cell tissue; an imaging system for imaging the applied microbubbles, wherein the abnormal cell tissue is located using the imaged, applied microbubbles, and wherein the imaging system detects and transmits imaging information of the applied microbubbles through a gaseous environment (or contactless procedure).

In an embodiment, the imaging system uses a multi-photon imaging (MPI) technique to image the applied microbubbles.

In an embodiment, the imaging system uses a technique selected from the group consisting of multi-photon imaging (MPI), optical coherence tomography (OCT), ultrasound, and a combination thereof, to image the applied microbubbles.

In an embodiment, the abnormal cell tissue is located adjacent to healthy pancreatic tissue.

In an embodiment, the abnormal cell tissue is located adjacent to healthy pancreatic, healthy brain, or other type of healthy tissue.

In an embodiment, the abnormal cell tissue is located below the surface of healthy tissue.

In an embodiment, the abnormal cell tissue is either in-vivo or ex-vivo.

In an embodiment, the imaging system images the applied microbubbles without the use of labels or markers.

In an embodiment, the imaging system uses harmonics (e.g., of third-order type) to image the applied microbubbles.

In an embodiment, the system further comprises a therapy system that applies therapy to the abnormal cell tissue. The therapy applied by the therapy system may be applied substantially simultaneously with the imaging by the imaging system.

Additional embodiments and additional features of embodiments for the method of using receptor-targeted microbubbles to locate abnormal cell tissue for therapy and system that uses receptor-targeted microbubbles to locate abnormal cell tissue for therapy are described below and are hereby incorporated into this section.

### BRIEF DESCRIPTION OF THE DRAWINGS

The foregoing summary, as well as the following detailed description, will be better understood when read in conjunction with the appended drawings. For the purpose of illustration only, there is shown in the drawings certain embodiments. It's understood, however, that the inventive concepts disclosed herein are not limited to the precise arrangements and instrumentalities shown in the figures. The detailed description will refer to the following drawings in which like numerals, where present, refer to like items.

FIG. 1A is a diagram illustrating the basic components of the lipid microbubble used in an embodiment. The specific targeted ligand (KTLLPTP) used was the selective for the plectin-1 receptor.

FIG. 1B is a diagram illustrating microbubble and focused femtosecond laser beam interaction. A THG signal is expected to be generated strongly from the liquid/air interface.

FIG. 1C is a diagram illustrating the synthesis of lipidated ligand performed by solid-phase technology using a Fmoc/tBu protection strategy (Scheme 1).

FIG. 2A is a schematic diagram illustrating the multiphoton microscope, in accordance with an embodiment.

FIG. 2B is a diagram illustrating a photograph of the microscope where both excitation laser sources are visible, in accordance with an embodiment.

FIGS. 3A-3B are diagrams illustrating lipid microbubbles conjugated with DiI. FIG. 3A shows an image taken by confocal microscopy where bubbles are dispersed and bound to a poly d-lysine cell culture plate with residual DiI washed away. FIG. 3B shows an image taken by multiphoton microscopy (using 1040 nm excitation laser and a 40× Nikon oil objective), specifically THG, where many unbound microbubbles are floating in a solution.

FIG. 3C is a diagram illustrating an emission spectrum from a 1560 nm multiphoton microscope displaying an emitted THG signal during microbubble imaging compared to the total pump laser.

FIGS. 4A-4C are diagrams illustrating targeted lipid microbubbles conjugated with DiI. FIG. 4A shows the THG signal from the bubbles only; FIG. 4B shows the fluorescence signal from the bubbles; and FIG. 4C shows a composite image of FIG. 4A, represented in red, and FIG. 4B, represented in green with the co-localized microbubbles represented with a yellow membrane.

FIGS. 5A-5D are diagrams illustrating pancreatic cancer cells with the targeted lipid microbubbles bound to the surface of the cells. FIG. 5A shows the THG signal from the bubbles only; FIG. 5B shows the fluorescence signal from the bubbles and cells; FIG. 5C shows a composite image of FIG. 5A, represented in red, and FIG. 5B, represented in green with the co-localized microbubbles represented in yellow; and FIG. 5D shows an image obtained from confocal microscopy for comparison.

FIG. 5E is a diagram illustrating a plot of an emission spectrum from a 1560 nm multiphoton microscope displaying an emitted THG signal during microbubble imaging compared to the total pump laser.

FIG. 6 is a flowchart illustrating an embodiment of a method of using receptor-targeted microbubbles to locate abnormal cell tissue for therapy, in accordance with an embodiment.

### DETAILED DESCRIPTION OF THE INVENTION

It is to be understood that the figures and descriptions of the present invention may have been simplified to illustrate elements that are relevant for a clear understanding of the present invention, while eliminating, for purposes of clarity, other elements found in a typical system that uses receptor-targeted microbubbles and typical method of using receptor-targeted microbubbles. Those of ordinary skill in the art will recognize that other elements may be desirable and/or required in order to implement the present invention. However, because such elements are well known in the art, and because they do not facilitate a better understanding of the present invention, a discussion of such elements is not provided herein. It is also to be understood that the drawings included herewith only provide diagrammatic representations of the presently preferred structures of the present invention and that structures falling within the scope of the present invention may include structures different than those shown in the drawings. Reference will be made to the drawings wherein like structures are provided with like reference designations.

Before explaining at least one embodiment in detail, it should be understood that the inventive concepts set forth herein are not limited in their application to the construction details or component arrangements set forth in the following description or illustrated in the drawings. It should also be understood that the phraseology and terminology employed herein are merely for descriptive purposes and should not be considered limiting.

It should further be understood that any one of the described features may be used separately or in combination with other features. Other invented devices, systems, methods, features, and advantages will be or become apparent to one with skill in the art upon examining the drawings and the detailed description herein. It is intended that all such additional devices, systems, methods, features, and advantages be protected by the accompanying claims.

For purposes of this disclosure, the terms “air” and “gas” may be used interchangeably.

There are potentially many different target areas/applications for the method/system described in this disclosure, although the pancreas is the focus herein for purposes of explanation only.

Embodiments include the combination of targeted microbubbles to cancer/abnormal cells and an imaging device with multiphoton imaging (MPI) and, optionally, in combination with other optical techniques such as optical coherence tomography (OCT), to accurately and rapidly assess the surgical margin for presence of unseen cancer in the operating room or other venues.

Embodiments are a unique solution for image-guided detection and surgery options in pancreatic or other types of cancer.

Embodiments may be used in real-time and may analyze the entire cut surface of a pancreas.

Pancreatic cancer is the 10^{th} most common cancer and 4^{th} highest cause of cancer death. The presence of a tumor at the surgical margin is the largest risk factor of poor survival. Surgeons currently palpate for a soft, normal area outside the hard tumor of the pancreas to determine the site of resection. Unfortunately, microscopic cancer cells cannot be palpated. Furthermore, cancerous cells at the cut surface cannot be seen by the naked eye. Embodiments herein solve this problem by a novel imaging system combined with targeted-microbubble technology to confirm or identify cancer (e.g., of residual type) in, for example, real-time.

The technology is the combination of targeted microbubbles to cancer cells and a novel imaging device to accurately and rapidly assess the surgical margin for presence of unseen cancer in the operating room. The device may be a portable multi-photon imaging (MPI) and optionally combined with other imaging technology such as optical coherence tomography (OCT) to visualize tissues with attached targeted microbubbles. MPI provides sub-cellular resolution and OCT provides rapid, wide-field, sub-surface imaging. Alternatively, OCT may be used alone, i.e. without the use of MPI. However, MPI can detect microbubbles with higher resolution.

The methodology is not limited only to pancreatic cancer and can be applied to, for example, general oncologic surgery.

Embodiments of the invention can be used in real-time during operation.

### 1. INTRODUCTION

Embodiments of the present invention utilize receptor-targeted lipid microbubbles to help ensure clean margins on remaining viable pancreatic tissue after the surgical removal of the tumor. Although ultrasound easily images microbubbles intravenously, in an external environment ultrasound requires a medium between the transducer and the object being imaged. Pressure on microbubbles from direct contact with ultrasound gel and a transducer can cause the bubbles to collapse or burst, leading to diagnostic inaccuracy. Moreover, this can become problematic when sterile environments need to be maintained. In order to increase simplicity and preserve accuracy, imaging modalities need to be explored that can detect microbubbles directly on a surface without the use of a contact medium. Multiphoton microscopy (MPM) has the ability to image contact-free, eliminating any concerns regarding contamination of a tissue surface. In addition, this procedure can be miniaturized into a hand-held probe, making the imaging device easily applicable to, for example, intraoperative settings where point-of-care diagnostics can be utilized quickly and efficiently.

Embodiments described herein provide successful imaging of microbubbles using, for example, a multi-photon microscope with compact femtosecond fiber lasers operating at, for example, 1560 nm and 1040 nm. In particular, the inventors were able to explore, via contact-free imaging, the binding of receptor-targeted lipid microbubbles in vitro on pancreatic tumor cell culture, using, for example, third-harmonic generation (THG). Compared to fluorescence detection, THG does not require an external marker and has a much larger dynamic range and thus a larger probing sensitivity. This technique has the potential to provide the accuracy and specificity required for detection of cancer in earlier stages, as well as inflammatory markers in the body. To show the capability of the technique, the detection of plectin-targeted lipid microbubbles was analyzed, as these receptors are overexpressed in pancreatic cancer cell lines. This method is fast and accurate, making it useful for the analysis of large-scale tissue samples. The detection of microbubbles using, for example, THG is a novel technique and could apply to a wide range of diagnostic applications.

### 2. PREPARATION AND SETUP OF AN EMBODIMENT

2.1. Preparation

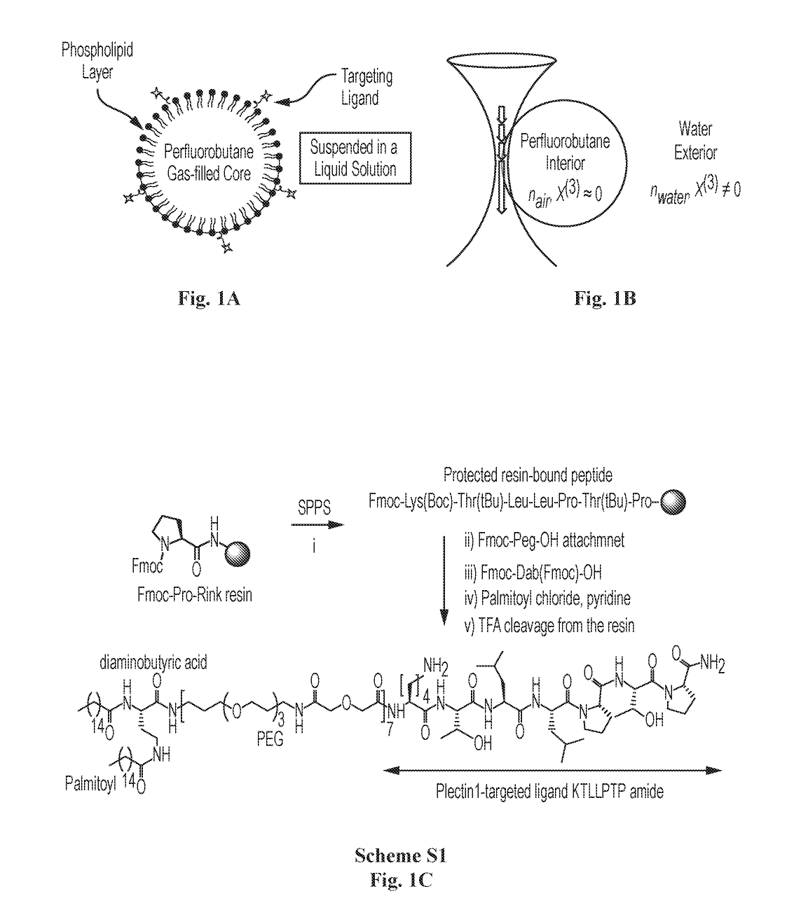

Plectin-1 was recently identified as a receptor biomarker to detect pancreatic ductal adenocarcinoma (PDAC). This receptor is identified in 100% of tested PDAC tumors and 60% of pre-invasive PanIN III lesions. Immunohistochemistry of human tissue has shown that Plectin-1 is not expressed by most normal tissue, with the exception of the skin and genitourinary tract. Plectin-1 specific ligand was panned from a phage display screen reported previously. The inventors have adopted a peptide ligand for specific targeting of PDAC in lipid microbubble imaging. A peptide was attached to the bis-palmitoyl lipid-like moiety via a short polyethyleneglycol spacer (extended span distance ˜140 Å). The specific peptide sequence was H-Lys-Thr-Leu-Leu-Pro-Thr-Pro-NH₂. The synthesis of lipidated ligand was performed by solid-phase technology using a Fmoc/tBu protection strategy (Scheme S1), as illustrated in FIG. 1C.

FIG. 1A is a diagram illustrating the basic components of the lipid microbubble used in an embodiment. The specific targeted ligand (KTLLPTP) used was the selective for the plectin-1 receptor. FIG. 1B is a diagram illustrating microbubble and focused femtosecond laser beam interaction. A THG signal is expected to be generated strongly from the liquid/air interface.

The lipid microbubble formulation is depicted in FIG. 1. The bubbles were prepared with a lipid composition containing dipalmitoylphosphatidylcholine (DPPC) (Genzyme, Cambridge, Mass., USA), 1,2-dipalmitoyl-sn-glycero-3-phosphate (monosodium salt) (DPPA) (Avanti Polar Lipids, Alabaster, Ala., USA), and lipidated ligand targeting pancreatic cancer cells. The lipid composition was dispersed in an excipient solution of phosphate-buffered saline (PBS), propylene glycol, and glycerol for a total lipid concentration of 1 mg mL⁻¹. From this lipid solution, 1.5 mL was pipetted into a 2 mL glass vial (Wheaton Industries, Millville, N.J.), and the air headspace was then replaced with decafluorobutane (DFB) gas (Fluoromed, Round Rock, Tex., USA). The microbubbles were then formed by mechanical agitation using a modified dental amalgamator (Lantheus Medical, New York, N.Y.), resulting in a microbubble distribution ranging predominantly from 1-10 microns with a concentration of 1-5×10⁹ microbubbles per mL of solution. The liquid microbubble is imaged using a tightly focused femtosecond laser beam, which is scanned across it. THG signal is expected to be generated strongly from the liquid/gas interface.

2.2. Cell Culture

The pancreatic cancer cell lines PANC-1 and MIA-PaCA2, which have amplified plectin expression, were grown in the University of Arizona Cancer Center using Dulbecco's Modified Eagle's Medium (DMEM) with 4.5 g/L glucose, L glutamine, and sodium pyruvate, supplemented with 10% fetal bovine serum and 1% penicillin-streptomycin. The cells were incubated in a 5% carbon dioxide, humidified atmosphere at 37° C. Cells were detached with trypsin and transferred onto poly-d-lysine coated glass bottom dishes (Mat Tek, Ashland, Mass.) followed by incubation for an additional 24 hours to insure adherence.

2.3. Multiphoton Microscope

Multi-photon (MP) imaging is a powerful technique that allows three dimensional mapping of samples that have a measurable nonlinear optical response such as second harmonic generation, third harmonic generation, or fluorescence induced by MP absorption. MP imaging (MPI) is currently an important tool for biological research and efforts are underway to turn this useful imaging technology into robust instruments for clinical applications. Lipid microbubbles are small, spherical structures formed by a thin lipid layer and contain a biocompatible gas (e.g., DFB) inside. These bubbles are typically dispersed in a liquid medium (solution) where they can attach to binding sites if their lipid membrane is functionalized with a suitable ligand.

There exists a transition from liquid phase to gas phase at the surface of the bubbles, stabilized by a thin lipid membrane. For that reason, ultrasound has been used to detect these bubbles due to the large scattering of ultrasound signal at their liquid/gas interfaces. In optics, third harmonic generation (THG) is a nonlinear optical effect, which arises from the third order nonlinear optical response of a material. THG has been shown to be very useful in label-free multiphoton imaging. Due to the Gouy phase shift in a tightly focused laser beam, the THG signal is generated only from interfaces where there is a change in the refractive index (or change in the third order nonlinear response). For that reason, THG has been shown to be useful in detecting these interfaces in biological tissues. Given the above, the inventors hypothesized that THG should be useful as a contactless detection of targeted lipid microbubbles given the sudden liquid/gas transition that these bubbles exhibit.

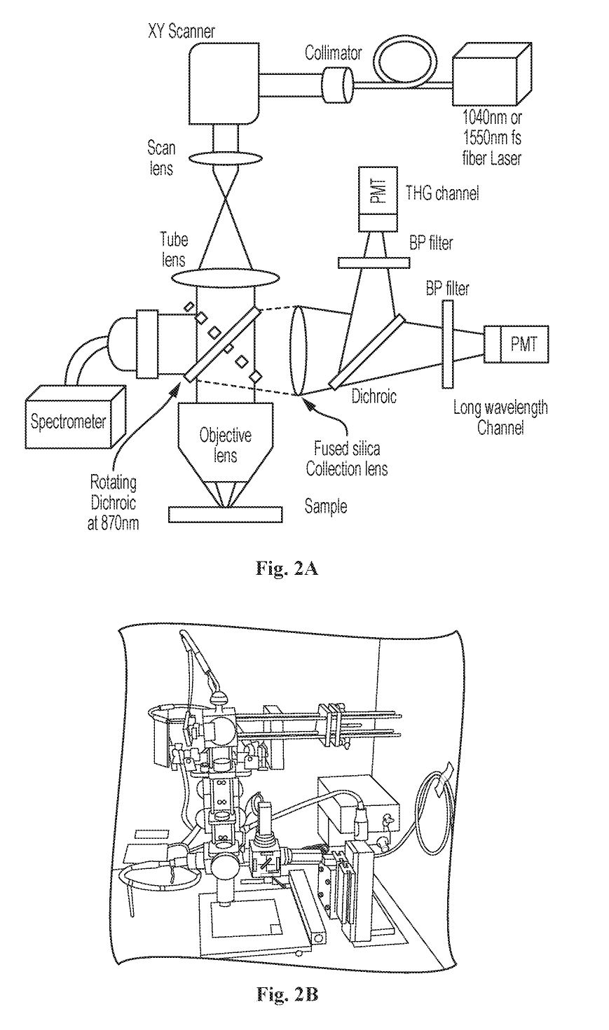

An exemplary new feature that the inventors added to a conventional microscope system is a new femtosecond laser operating at 1040 nm which emits ˜70 mW average power at ˜8 MHz repetition rate and ˜100 fs pulse duration. The microscope can accommodate both 1560 nm and 1040 nm without changing the optics in the excitation beam path. The addition of the 1040 nm laser allows excitation of the marker dye, DiI, that embodiments use to co-localize the fluorescence marker with the THG signal (described below) to confirm that THG is indeed detected from the liquid/gas interface of the bubbles. The dichroic filter and bandpass filter in front of the photomultiplier tube (PMT) for THG signal detection are changed to match the new excitation wavelength respectively. Specifically, a 345 nm bandpass filter (˜20 nm pass band) is used in front of the PMT to detect the THG signal. A pump filter (Semrock) is also used to remove pump laser light from reaching the PMTs. A 520 nm bandpass filter is used with the 1560 nm excitation laser. The diagram, of the microscope can be seen in FIGS. 2A-2B.

FIG. 2A is a schematic diagram illustrating the multiphoton microscope, in accordance with an embodiment. The system has a THG spot size of ˜350 nm and an axial resolution of 2 μm (with a Nikon 40× objective, NA 13) at the excitation wavelength of 1040 m. The system has a THG spot size of ˜525 nm and an axial resolution of 3 μm (with a Nikon 40× objective, NA 1.3) at the excitation wavelength of 1560 nm. FIG. 2B is a diagram illustrating a photograph of the microscope where both excitation laser sources are visible, in accordance with an embodiment. The 1560 nm laser is the gray box on top and the 1040 nm laser source is the black box at the bottom. An Ocean Optics spectrometer (350 nm-1100 nm detection range) is integrated into the multiphoton microscope for measuring the optical spectrum of the multiphoton excited signals (black box with blue input fiber on the left).

### 3. RESULTS OF AN EMBODIMENT

3.1 Theoretical Description of TUG Generated MPNI Signal

Imaging results for these microbubbles indicate that MPM signals are dominated by THG at the interface resulting from a difference in bulk third-order susceptibilities. To model this, embodiments approximate the DFB interior as air:

(nᵢₙₜₑᵣᵢₒᵣ(λ)≈n_{cir}(λ),χ⁽³⁾≈0—linear, dispersive and isotropic)

and the exterior as water:

(nₑₓₜₑᵣᵢₒᵣ(λ)≈n_{water}(λ),χ⁽³⁾≈χ_{water}⁽³⁾—nonlinear, dispersive, and isotropic).

Because the boundary lipid monolayer is very small in scale (<10 nm) and because live cell walls (water inside and outside) have not generated an appreciable MPM signal in the configuration, embodiments neglect any nonlinear polarizability of the lipid layer itself. The fact that embodiments are predominantly resolving interfaces is explained by the Gouy phase shift. If the focus of the laser beam is not at an interface, the Gouy phase shift cancels THG generated before the focus with TUG generated after the focus. This occurs because the coupling between the fields is the same strength before and after the focus but is 180° out of phase. Thus, the signal that is obtained in this embodiment is dominated by spatial variation of χ⁽³⁾. At interfaces, the THG field generated before the focus does not get converted back to the fundamental. The strength of the generated signal in the moderate focusing case (½<NA<1) has been reported as:

$${I}_{3 ω} ∝ κ {\vert \frac{{χ}_{water}^{( 3 )}}{{n}_{3 ω} ( {n}_{3 ω} - {n}_{ω} )} \vert}^{2} {I}_{ω}^{3}$$

3.2 Imaging of Lipid Microbubbles Only Using Confocal Microscopy and MPM

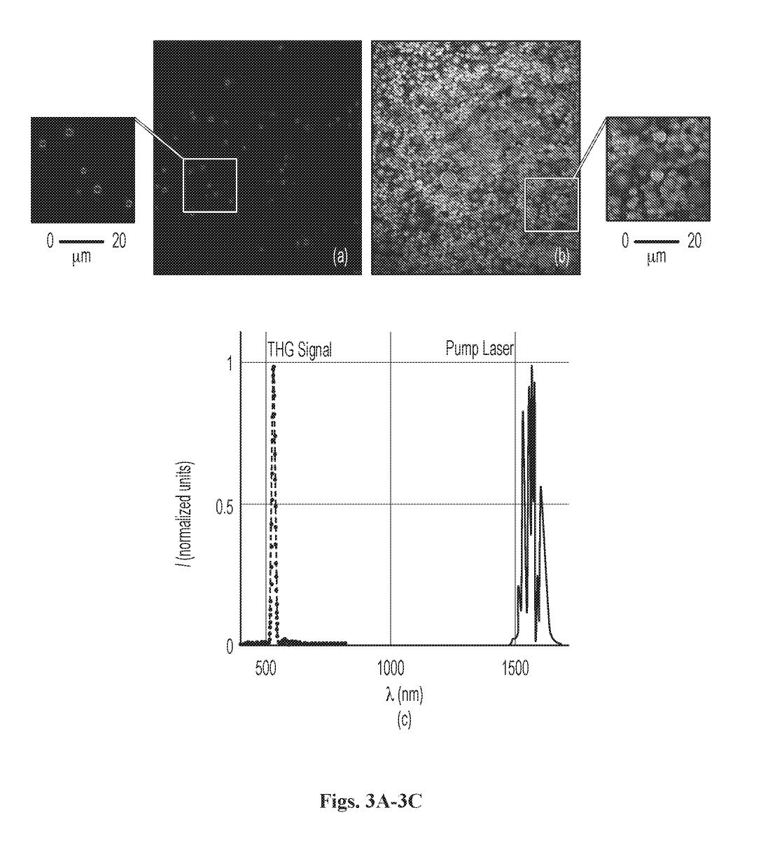

FIGS. 3A and 3B are diagrams illustrating lipid microbubbles conjugated with DiI. FIG. 3A shows an image taken by confocal microscopy where bubbles are dispersed and bound to a poly d-lysine cell culture plate with residual DiI washed away. FIG. 3B shows an image taken by multiphoton microscopy (using 1040 nm excitation laser and a 40× Nikon oil objective), specifically THG, where many unbound microbubbles are floating in a solution. FIG. 3C is a diagram illustrating an emission spectrum from a 1560 nm multiphoton microscope displaying an emitted THG signal during microbubble imaging compared to the total pump laser.

The unique interface of the liquid-to-gas transition of the lipid microbubble generates a THG signal that is demonstrated in FIGS. 3A-3C. FIG. 3B shows the THG signal generated from the lipid microbubbles through the use of a filter that separates the THG signal into the corresponding PMT detector. FIG. 3A shows lipid microbubbles bound to a surface imaged with confocal microscopy (for comparison purpose). The microbubbles could not be imaged with confocal microscopy in a solution, as in FIG. 3B, because the solution contains residual DiI molecules that do not insert into the membrane of the bubbles. Since confocal microscopy only detects the fluorescent DiI signal from the bubble membrane, the resolution is not high enough to separate the signal from the DiI molecules on the microbubble membrane from the residual DiI in the solution. Therefore, to obtain FIG. 3B, the inventors allowed the lipid microbubbles to bind to the bottom of a petrie dish and then wash the remaining bubble solution off the dish so that only the bound microbubbles were imaged. Thus allowing us to obtain clear bubble images with confocal microscopy because there was no residual DiI impeding the signal. This residual DiI makes no difference in the MPM image since MPM is detecting only the liquid to gas phase change of the bubble due to the THG signal as observed in the emission spectrum in FIG. 3C. Since the image obtained from MPM can be obtained label-free, it makes it a promising solution for lipid microbubble detection.

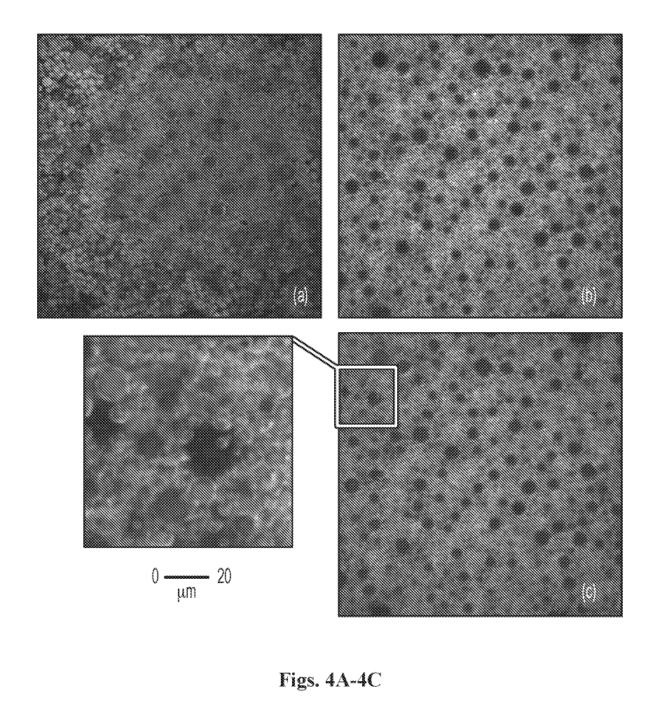

To verify that the signal obtained from MPM was specifically THG, the inventors employed another technique/embodiment with a lipid microbubble solution conjugated with DiI to prove co-localization between the fluorescent light channel and THG channel. The inventors used the 1040 nm laser to image the bubble solution with a 538 nm dichroic filter as well as a 345 nm bandpass THG filter. The beam split through the filters was such that the DiI signal appeared only in the fluorescent light channel and the THG signal appeared only in the third harmonic generation channel. FIGS. 4A-4C show the images obtained from this embodiment. Namely, FIGS. 4A-4C are diagrams illustrating targeted lipid microbubbles conjugated with DiI. FIG. 4A is an image of the THG channel, FIG. 4B is an image of the fluorescent light channel, and FIG. 4C is an image of FIG. 4A and FIG. 4B overlaid with THG represented in red and fluorescent light represented in green. Co-localization of the microbubbles causes the bubble membrane to appear yellow.

3.3 Imaging of Targeted and Labeled Lipid Microbubbles Using Confocal Microscopy

To verify lipid microbubble binding to the cell strains, the inventors first imaged with confocal microscopy. Twenty-four hours after the cells had been plated, the cells were rinsed with Dulbecco's phosphate buffered saline (DPBS) to remove debris from the microenvironment. Then the cells were incubated in DPBS for 30 minutes supplemented with 5 microliters of calcein dye in addition to 100 microliters of the lipid microbubbles conjugated with DiI. Due to the buoyancy of the microbubbles, the cells were inverted for this period to maximize exposure of the cells to the microbubbles. After the incubation period, the cells were washed with DPBS to remove any unbound microbubbles and were then maintained in media for imaging.

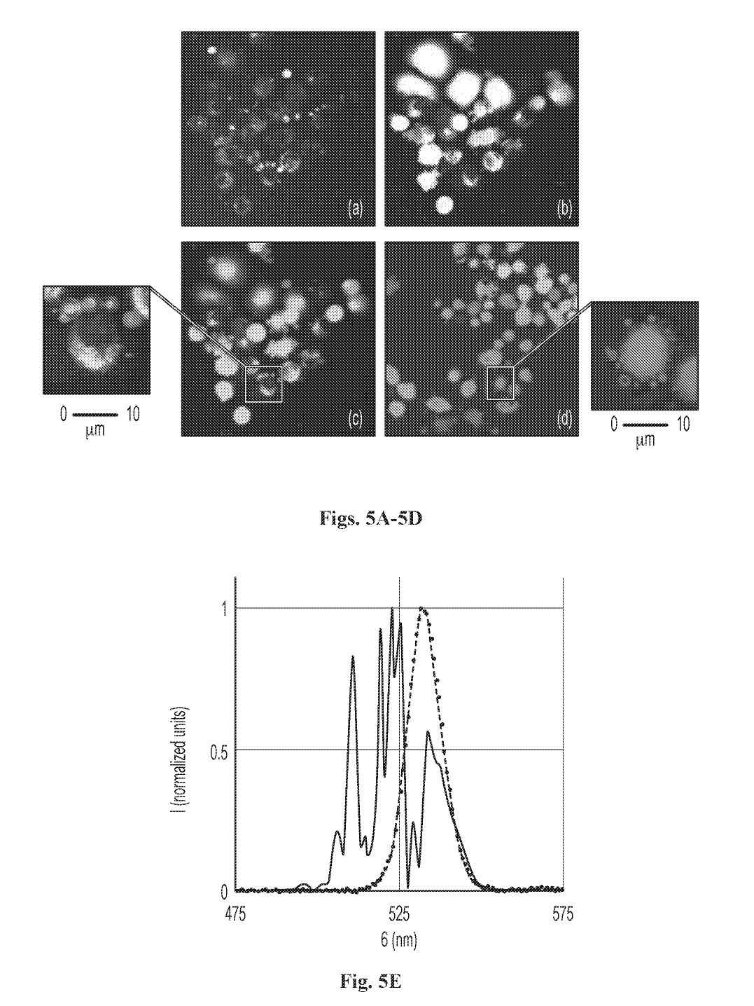

The conjugation was finally visualized under the AZCC Leica SP5 confocal microscope (Leica Microsystems, Buffalo Grove, Ill.) with a 63× oil immersion objective captured at 2048×2048 pixels to obtain a field of view of 246.03×246.03 um. The visible light wavelength lasers used were the 50 mW Argon laser (458, 477, 488, and 514 nm) and the 1 mW Helium Neon Laser (543 nm) to capture the spectrum of the calcein (ex495/em515) fluorescing the cytoplasm of the living cells and DiI (ex549/em565) conjugated to the lipid microbubble membrane. FIGS. 5A-5D displays the evident binding of the plectin-targeted lipid microbubbles to the pancreatic cancer cells.

3.4 Multiphoton Imaging of Microbubbles

The technique of detecting lipid microbubbles through THG can be proven through co-localization of the DiI fluorescence emitted from the microbubble membrane and the independent THG signal resulting from the phase change of the laser signal at the gas-liquid interface. In order to obtain separate DiI and THG signals, filters need to be added to the multiphoton microscope so that the two filters split the beam emitted from the laser. The beam split through the filters would be such that the DiI signal appears only in the fluorescent light channel and the THG signal appears only in the third harmonic generation channel. These two channels can then be combined to show the DiI signal from the bubble membrane and the THG signal from the phase change overlap thereby demonstrating dual and independent detection of label-free microbubble membranes.

The same procedure referenced above (Section 3.3) was used to prepare the cells for MPM imaging. The cells were visualized under the 1040 nm multiphoton microscope with a water immersion 40× objective (0.75 NA) with two filters to demonstrate THG generation can image plectin targeted lipid microbubbles. The filters used in the system were a 538 nm dichroic filter and the 345 nm bandpass THG filter. The 538 nm dichroic filter was used to send the fluorescent emission signals of the DiI on the bubbles and the calcein on the cells to one PMT detector. The THG filter was used to send the emission signal from the phase change of the bubbles to the other PMT detector. FIGS. 5A-5D displays the images obtained from the two separate filters, a composite image of the filter combination, and an image from confocal imaging to compare to the final MPM image. Specifically, FIG. 5A represents the THG signal, FIG. 5B represents the fluorescent DiI signal, and FIG. 5C is the composite image of FIG. 5A (represented in red) and FIG. 5B (represented in green) so that the co-localized bubbles appear yellow. The presence of the yellow bubbles in FIG. 5C demonstrates co-localization and defines THG as a method of detection for lipid microbubbles. FIG. 5D shows an image obtained from confocal microscopy for comparison. FIG. 5E is a diagram illustrating a plot of an emission spectrum from a 1560 nm multiphoton microscope displaying an emitted THG signal during microbubble imaging compared to the total pump laser.

### 4. DISCUSSION AND CONCLUSION

The contact-free detection of targeted lipid microbubbles was explored using, for example, the THG imaging modality of multiphoton microscopy. This novel technique can be used for a wide variety of applications. The main application of this work stems from the large dynamic range as well as high probing sensitivity of THG, making it a practical imaging method for earlier detection of cancer and inflammatory markers in the body. Moreover, the development of this device into a hand-held probe could provide a method of receptor-targeted imaging of the entire surface of a surgical margin at the point-of-care during the operation, which is currently not possible, and which should improve cancer-free survival and reduce the overall cost of care.

Embodiments described in this disclosure are capable of imaging microbubbles on or below the surface of healthy tissue.

Monochromic antibodies have been utilized on the surface of microbubbles. However, monochromic antibodies are not stable when they are adhered to the surface of a microbubble. To overcome this deficiency, a small molecule regime, something that doesn't denature, has been contemplated. The change to a small molecule approach has facilitated the targeting of cancer/abnormal cells.

Embodiments described herein may be performed quickly without having to send samples to pathology.

Previous techniques use ultrasound gel or other contact media to apply an ultrasound probe onto tissue. However, the use of the ultrasound gel or contact media is, inter alia, not compatible with a sterile environment.

Embodiments provide a contactless imaging solution to transmit through air/gas so that contact of any surface would not be required for imaging purposes. An MPI imaging technique never needs to touch tissue, i.e., it can be, for example, a couple of centimeters away to avoid violating the sterile field. With contactless imaging techniques such as MPI, embodiments would not need a gel or interface for the imaging system.

The imaging system may be contained within a miniaturized, handheld, smartphone-sized or smaller, enclosure that operates without contacting the tissue and that can scan fast enough to scan an entire cut surface to see if there were residual or any other type cancer cells.

In the field, no microbubbles have ever been used to target cancer cells or solid organ surfaces. Current imaging techniques do not teach that microbubbles can be used on solid organs bypassing the vascular system.

Embodiments are directed to targeting of microbubbles in conjunction with imaging (e.g., multimodal imaging techniques such as MPI) and, optionally, point-of-care diagnostics and/or intra-operative procedures.

Embodiments herein may be used to treat abnormal cells on or within the pancreas, brain, or anywhere in the body, to remove the tumor-containing portion and where maximum amount of tissue is desired to be left intact in the patient. Embodiments provide a process which includes selective guidance for surgical removal so that there is a clean margin at the end where the tumor is removed and where maximum amount of tissue is left intact in patient. Embodiments provide a process which includes confirmation of a clean, cancer or disease-free margin on specimens after resection from the body, including its imaging or analysis ex-vivo. Much of what the inventors propose is on the resected specimen and, therefore, the process can be done in-vivo as well as ex-vivo.

Embodiments provide techniques of identifying unseen cancer during surgery or at other times. These techniques facilitate an efficient removal of abnormal cell tissue for long-term survival benefits. The novel imaging techniques can be used in various settings including operating rooms in a way that allows us to look at an entire surface area and not just a tiny piece of the body, both inside the body as well as on tissue or cells removed from the body.

Embodiment may utilize any contactless imaging such as MPI, OCT, or fluorescence antibody markers on microbubbles which may detect using any camera that can detect fluorescence.

Embodiments can utilize, for example, second or third harmonics to image microbubbles and which do not require exogenous labels. Once targeted/adhered, an additive to the microbubbles is not required. Another chemical is not needed to be added to identify the microbubbles. The microbubbles can be identified using, for example, 3rd harmonics alone. Third harmonics also allows the sub-surface tissue to be imaged simultaneously for tumor(s), beyond the imaging of bubbles on cell surfaces.

Embodiments may provide point-of-care diagnostics with sufficient detection anytime, e.g., during surgery, at bedside, etc.

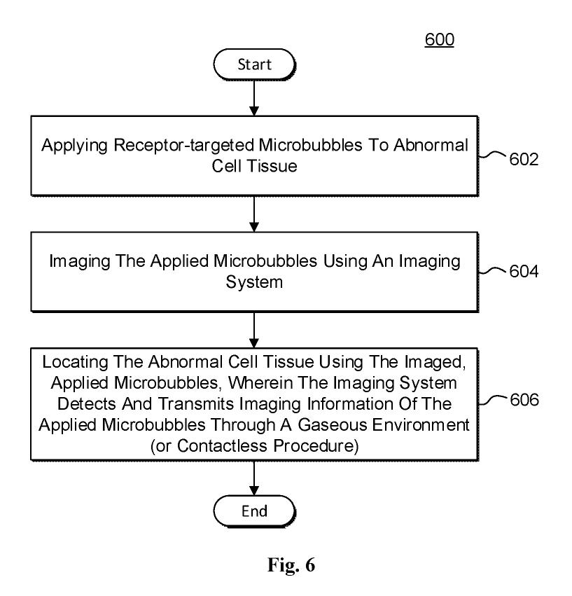

Embodiments are directed to a method of using receptor-targeted microbubbles to locate abnormal cell tissue for therapy. FIG. 6 is a flowchart illustrating an embodiment of a method 600 of using receptor-targeted microbubbles to locate abnormal cell tissue for therapy, in accordance with an embodiment. In an embodiment, the method comprises: applying receptor-targeted microbubbles to abnormal cell tissue (block 602); imaging the applied microbubbles using an imaging system (block 604); and locating the abnormal cell tissue using the imaged, applied microbubbles, wherein the imaging system detects and transmits imaging information of the applied microbubbles through a gaseous environment (or contactless procedure) (block 606).

In an embodiment, the imaging is performed by the imaging system using a light source (such as a laser).

In an embodiment, the imaging is performed by the imaging system using a multi-photon imaging (MPI) technique.

In an embodiment, the imaging is performed by the imaging system using a technique selected from the group consisting of multi-photon imaging (MPI), optical coherence tomography (OCT), ultrasound, and a combination thereof.

In an embodiment, the abnormal cell tissue is located adjacent to healthy pancreatic tissue.

In an embodiment, the abnormal cell tissue is located adjacent to healthy pancreatic, healthy brain, or other type of healthy tissue.

In an embodiment, the abnormal cell tissue is located below the surface of healthy tissue.

In an embodiment, the abnormal cell tissue is either in-vivo or ex-vivo.

In an embodiment, the imaging of the applied microbubbles is performed without the use of labels or markers.

In an embodiment, the imaging of the applied microbubbles is performed using harmonics (e.g., third-order harmonics).

In an embodiment, the method further comprises applying therapy to the abnormal cell tissue. The imaging and applying of therapy may be performed substantially simultaneously.

Embodiments are also directed to a system that uses receptor-targeted microbubbles to locate abnormal cell tissue for therapy. In an embodiment, the system comprises: an application system that applies receptor-targeted microbubbles to abnormal cell tissue; an imaging system for imaging the applied microbubbles, wherein the abnormal cell tissue is located using the imaged, applied microbubbles, and wherein the imaging system detects and transmits imaging information of the applied microbubbles through a gaseous environment (or contactless procedure).

In an embodiment, the imaging system uses a multi-photon imaging (MPI) technique to image the applied microbubbles.

In an embodiment, the imaging system uses a technique selected from the group consisting of multi-photon imaging (MPI), optical coherence tomography (OCT), ultrasound, and a combination thereof, to image the applied microbubbles.

In an embodiment, the abnormal cell tissue is located adjacent to healthy pancreatic tissue.

In an embodiment, the abnormal cell tissue is located adjacent to healthy pancreatic, healthy brain, or other type of healthy tissue.

In an embodiment, the abnormal cell tissue is located below the surface of healthy tissue.

In an embodiment, the abnormal cell tissue is either in-vivo or ex-vivo.

In an embodiment, the imaging system images the applied microbubbles without the use of labels or markers.

In an embodiment, the imaging system uses harmonics (e.g., of third-order type) to image the applied microbubbles.

In an embodiment, the system further comprises a therapy system that applies therapy to the abnormal cell tissue. The therapy applied by the therapy system may be applied substantially simultaneously with the imaging by the imaging system.

Although embodiments are described above with reference to the application of microbubbles to abnormal cell tissue (and the locating of the abnormal cell tissue using the imaged, applied microbubbles), the microbubbles may alternatively or additionally be applied to a collection of abnormal cells (to similarly locate the collection of abnormal cells using the imaged, applied microbubbles). Such alternatives are considered to be within the spirit and scope of the present invention, and may therefore utilize the advantages of the configurations and embodiments described above.

The method steps in any of the embodiments described herein are not restricted to being performed in any particular order. Also, structures or systems mentioned in any of the method embodiments may utilize structures or systems mentioned in any of the device/system embodiments. Such structures or systems may be described in detail with respect to the device/system embodiments only but are applicable to any of the method embodiments.

Features in any of the embodiments described in this disclosure may be employed in combination with features in other embodiments described herein, such combinations are considered to be within the spirit and scope of the present invention.

The contemplated modifications and variations specifically mentioned in this disclosure are considered to be within the spirit and scope of the present invention.

More generally, even though the present disclosure and exemplary embodiments are described above with reference to the examples according to the accompanying drawings, it is to be understood that they are not restricted thereto. Rather, it is apparent to those skilled in the art that the disclosed embodiments can be modified in many ways without departing from the scope of the disclosure herein. Moreover, the terms and descriptions used herein are set forth by way of illustration only and are not meant as limitations. Those skilled in the art will recognize that many variations are possible within the spirit and scope of the disclosure as defined in the following claims, and their equivalents, in which all terms are to be understood in their broadest possible sense unless otherwise indicated.

1. A method of using receptor-targeted microbubbles to locate abnormal cell tissue for therapy, the method comprising:
applying receptor-targeted microbubbles to abnormal cell tissue;
imaging the applied microbubbles using an imaging system; and
locating the abnormal cell tissue using the imaged, applied microbubbles, wherein the imaging system detects and transmits imaging information of the applied microbubbles through a gaseous environment.

2. The method of claim 1, wherein the imaging is performed by the imaging system using a light source.

3. The method of claim 1, wherein the imaging is performed by the imaging system using a multi-photon imaging (MPI) technique.

4. The method of claim 1, wherein the imaging is performed by the imaging system using a technique selected from the group consisting of multi-photon imaging (MPI), optical coherence tomography (OCT), ultrasound, and a combination thereof.

5. (canceled)

6. The method of claim 1, wherein the abnormal cell tissue is located adjacent to healthy pancreatic, healthy brain, or other type of healthy tissue.

7. The method of claim 1, wherein the abnormal cell tissue is located below the surface of healthy tissue.

8. The method of claim 1, wherein the abnormal cell tissue is either in-vivo or ex-vivo.

9. The method of claim 1, wherein the imaging of the applied microbubbles is performed without the use of labels or markers.

10. The method of claim 1 where the imaging of the applied microbubbles is performed using harmonics.

11. (canceled)

12. The method of claim 1, further comprising applying therapy to the abnormal cell tissue.

13. The method of claim 12, wherein the imaging and applying of therapy are performed substantially simultaneously.

14. A system that uses receptor-targeted microbubbles to locate abnormal cell tissue for therapy, the system comprising:
an application system that applies receptor-targeted microbubbles to abnormal cell tissue; and
an imaging system for imaging the applied microbubbles, wherein the abnormal cell tissue is located using the imaged, applied microbubbles, and wherein the imaging system detects and transmits imaging information of the applied microbubbles through a gaseous environment.

15. The system of claim 14, wherein the imaging system uses a multi-photon imaging (MPI) technique to image the applied microbubbles.

16. The system of claim 14, wherein the imaging system uses a technique selected from the group consisting of multi-photon imaging (MPI), optical coherence tomography (OCT), ultrasound, and a combination thereof, to image the applied microbubbles.

17. (canceled)

18. The system of claim 14, wherein the abnormal cell tissue is located adjacent to healthy pancreatic, healthy brain, or other type of healthy tissue.

19. The system of claim 14, wherein the abnormal cell tissue is located below the surface of healthy tissue.

20. The system of claim 14, wherein the abnormal cell tissue is either in-vivo or ex-vivo.

21. The system of claim 14, wherein the imaging system images the applied microbubbles without the use of labels or markers.

22. The system of claim 14, wherein the imaging system uses third-order harmonics to image the applied microbubbles.

23. The system of claim 14, further comprising a therapy system that applies therapy to the abnormal cell tissue.

24. The system of claim 23, wherein the therapy applied by the therapy system is applied substantially simultaneously with the imaging by the imaging system.
